(19) United States
(12) Patent Application Publication
(10) Pub. No.: US 2025/0279158 A1
(43) Pub. Date: Sep. 4, 2025

(54) COMPUTER-ASSISTED METHOD AND SYSTEM FOR EVALUATING AND MODIFYING IMMUNOGENICITY OF PROTEIN SEQUENCES USING A PROTEIN LARGE LANGUAGE MODEL

(71) Applicant: Lurong Pan, Vestavia Hill, AL (US)

(72) Inventor: Lurong Pan, Vestavia Hill, AL (US)

(21) Appl. No.: 18/591,951

(22) Filed: Feb. 29, 2024

(51) Int. Cl. G16B 15/30; G16B 40/20; G16B 40/30; G16B 50/30

(52) U.S. Cl. CPC G16B 15/30; G16B 40/20; G16B 40/30; G16B 50/30

(57) ABSTRACT

The embodiment discloses a computer-assisted method for evaluating and modifying immunogenicity, as well as related computer systems and storage media. The method includes using unsupervised learning of a protein large language model on all human sequences and a supervised deep learning neural networks to establish a predictive scoring model for the humanness score of peptide chains based on the data from the model training dataset to achieve classification between human and non-human species. This involves cutting protein sequences into all possible peptide chains of a preset length using a dynamic window method and importing these chains into the predictive scoring, thereby evaluating their immunogenicity in terms of humanness score. Peptide chains with scores above a certain threshold undergo all possible single-point virtual mutations to generate a set of modified peptide chains which are then reassessed using the model, selecting those with scores above the threshold of humanness for further consideration.

### TECHNICAL FIELD

Embodiments of the present disclosure generally relate to computer-assisted methods and systems and more particularly relate to evaluating and modifying immunogenicity of protein sequences using a protein large language model.

### BACKGROUND

Generally, the development of therapeutic proteins often encounters the challenge of immunogenicity. The immune system may recognize and respond to therapeutic proteins as foreign, leading to reduced efficacy or adverse immune reactions. Therefore, assessing and modifying the immunogenicity of protein sequences is a critical step in the development of therapeutic proteins.

Additionally, there are various technical problems with the evaluation of immunogenicity in the prior art. In the existing technology, evaluating immunogenicity relies on experimental approaches, which are time-consuming, expensive, and may not comprehensively predict the immunogenicity of all possible peptide chains within a protein sequence.

To address these challenges, artificial intelligence, particularly supervised and unsupervised learning using protein language model techniques, offers promising solutions. By leveraging large datasets, such as the Immune Epitope Database (IEDB), Observed Antibody Space (OAS) database and other publication sources. deep learning models can predict the probability difference between human and non-human protein or peptide sequences with both supervised and unsupervised learning algorithm. These predictive abilities are crucial for evaluating the immunogenicity of peptide chains within protein sequences.

Therefore, there is a need to provide a method and related systems for rapidly and comprehensively screening potential immunogenic peptide chains from protein sequences, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

An aspect of the present disclosure provides a computer-assisted method for immunogenicity assessment and modification, as well as related computer systems and storage media is provided. The computer-assisted immunogenicity assessment method may quickly and comprehensively screen the rapidly screening peptide chain sequences that may cause immunogenic reactions, and accelerating the development of therapeutic proteins is disclosed.

Another aspect of the present disclosure provides a method for computer-assisted evaluating immunogenicity of protein sequences using a pre-trained protein sequence large language model. Further, it is characterized by establishing a predictive scoring model for the protein sequences after processing them into all possible peptide chains of a preset lengths, thereby evaluating their immunogenicity.

Another aspect of the present disclosure is storing peptide chains whose humanness probability scores between 1 and 0, 1 as human, 0 as non-human, as determined by the protein large language model, are smaller than or equal to a first preset threshold between 0.5-0.8. Marking cutting sites on the protein sequence where the humanness scores are smaller than or equal to a first preset threshold.

Yet another aspect of the present disclosure is obtaining protein sequences includes translating protein sequences into FASTA format files and importing these files into the protein large language model with different tokenization methods. Tokenization of protein sequences involves breaking down the sequences into manageable units (tokens) such as amino acids, motifs, or domains. These tokens serve as the input for various computational models, including Single Amino Acid Tokenization, K-mer Tokenization, N-gram Tokenization, Motif-based Tokenization, Domain-based Tokenization.

Additionally, the protein large language model is part of a supervised deep learning model to classify between immunogenic and non-immunogenic species, which includes one of, or a combination of Convolutional Neural Network (CNN), Recurrent Neural Network (RNN), Graph Neural Network (GNN), and Variational Autoencoders (VAEs), and Transformer models, all tailored for analyzing protein sequences.

Further, a computer-assisted method for modifying immunogenicity of protein sequences using a protein large language model, characterized by: Implementing the method for evaluating immunogenicity as described in claims 1 to 5; Identifying, via the protein large language model that includes CNN, RNN, GNN, VAE and Transformer, peptide chains whose humanness scores are smaller than or equal to a second preset threshold; Performing all possible single-point virtual mutations on these identified peptide chains to generate a set of modified peptide chains; Selecting modified peptide chains whose humanness probability scores are less than the second preset threshold.

Yet another aspect of the present disclosure is to provide a computer system for evaluating and/or modifying immunogenicity of protein sequences, characterized by including a processor and a memory connected to the processor, wherein the memory stores a program executable by the processor to implement the method for evaluating and/or modifying immunogenicity using a protein large language model. The pretrained protein large language model includes a combination of supervised training with one of, or an ensemble of CNN, RNN, GNN, VAEs and Transformer models for comprehensive protein sequence analysis.

Yet another aspect of the present disclosure is to provide a computer-readable storage medium, characterized by storing a computer program, which, when executed by a processor, implements the method for evaluating and/or modifying immunogenicity of protein sequences using a protein large language model. The protein large language model includes one or ensemble models of CNN, RNN, GNN, VAEs and Transformer as part of its deep learning architecture.

The present disclosure describes a sophisticated computer-assisted method for evaluating and modifying the immunogenicity of protein sequences, leveraging the capabilities of a protein Large Language Model (LLM). The method involves:
Deep Learning Integration: Utilizing a deep learning neural network that integrates a protein LLM, along with architectures like Convolutional Neural Network (CNN), Recurrent Neural Network (RNN), Graph Neural Network (GNN), Variational Autoencoders (VAEs) and Transformer models, specifically designed for protein sequence analysis.
Extensive unsupervised Pre-Training and supervised Training: Implementing extensive pre-training of the protein LLM on over 3.5 billions species-specific protein sequences, followed by additional training with data from various sources for enhanced predictive accuracy.

Protein Sequence Processing: Employing a moving window method to process protein sequences into peptide chains, ensuring comprehensive evaluation of potential immunogenic sequences.

Immunogenicity Evaluation and Modification: Scoring peptide chains for humanness likelihood and modifying those with high immunogenic potential through single-point virtual mutations, re-evaluating these to select chains with reduced immunogenicity.

Advanced Evaluation Techniques: Utilizing a range of sophisticated evaluation matrices, including AUC, Top(n), Precision, and Recall, to ensure a thorough and accurate assessment of the model's performance.

Implementation Support: The method is supported by a comprehensive computer system and a computer-readable storage medium, facilitating the implementation of this advanced immunogenicity evaluation and modification process.

The present invention represents a significant advancement in the field of therapeutic protein development, offering an efficient, accurate, and comprehensive method for immunogenicity assessment and modification. The application of advanced AI techniques, particularly the protein LLM, underscores the potential of this method to enhance the safety and efficacy of therapeutic proteins, with broad applications in biotechnology and pharmaceuticals.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 illustrates a schematic flow diagram 100 of steps of a computer-assisted method for immunogenicity assessment, in accordance with an embodiment of the present disclosure.

FIG. 2 illustrates a detailed view of system architecture 200, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

Technical Problem: The technical problem addressed by this invention is to provide a method and related systems for rapidly and comprehensively screening potential immunogenic peptide chains from protein sequences. The method aims to enhance the speed and efficiency of therapeutic protein development, overcoming the limitations of traditional experimental approaches.

Technical Solution: To solve the aforementioned problem, the present disclosure introduces a computer-assisted method that utilizes deep learning neural networks to establish a predictive scoring model. Further, the present model assesses the humanness score of peptide chains, thereby evaluating the immunogenicity of peptide chains. The method includes steps for cutting protein sequences into peptide chains, the length of the chain would be varied from 6-12, scoring their humanness likelihood score, and modifying peptide chains with high immunogenic potential through virtual mutations. The modified chains are then re-evaluated to select those with reduced immunogenicity. This approach offers a rapid, efficient, and comprehensive method for immunogenicity assessment and modification, leveraging the power of artificial intelligence and deep learning. The present specification provides the following technical solutions: A computer-assisted method for immunogenicity assessment, including the following steps: —Based on the data from the model training dataset, establish a predictive scoring model for the immunogenicity likelihood of each peptide chains using a deep learning neural network.
Obtain protein sequences and cut them into all possible peptide chains of a preset length using a dynamic sliding window method.
Sequentially import each cut peptide chain into the predictive scoring model, scoring each chain's immunogenicity as the score

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Embodiments in this document are described in a progressive manner, where similar aspects of different embodiments can be referred to interchangeably, focusing mainly on the differences between each embodiment. Particularly, the embodiments related to the computer system and the readable storage medium are described succinctly as they correspond to the method's embodiments, and for details, one can refer to the method's embodiments.

Further, the present disclosure provides a computer-assisted method for evaluating and modifying immunogenicity, as well as related computer systems and storage media. The core of this invention is the establishment of a predictive humanness scoring model for the peptide chains using deep learning neural networks.

Furthermore, the method comprises the following steps:
Data Preparation: Protein sequences are obtained and cut into all possible peptide chains of a preset length using a dynamic window method. These peptide chains are then imported into the predictive scoring model.
Model Development: A deep learning neural network, incorporating a protein Large Language Model (LLM), is used to establish the predictive scoring model. This model integrates various architectures, including CNN, RNN, GNN, VAE and Transformer models. Further, for the base model development, the protein LLM is pre-trained on a massive dataset comprising over 3.5 billion species-specific protein sequences. This extensive pre-training ensures that the model has a broad and deep understanding of protein structures and functions. The model is further trained with data from various sources, to ensure comprehensive coverage and accuracy in predicting the humanness score.
Scoring and Evaluation: Each peptide chain is scored for its humanness score. Peptide chains with scores above a certain threshold are identified as potential immunogenic peptides.
AUC (Area Under the Curve): Represents the degree of separability in ROC analysis; higher AUC indicates better model performance.
Top(n) and Top(n+2): Performance metrics indicating the model's accuracy in predicting the top n or n+2 results.
Precision: The ratio of correctly predicted positive observations to the total predicted positives.
Recall: The ratio of correctly predicted positive observations to all observations in the actual class.
Modification and Reevaluation: Identified peptide chains undergo all possible single-point virtual mutations to generate a set of modified peptide chains. These modified chains are then re-scored using the model.

Peptide chains whose modified scores fall above the threshold are selected for further consideration. The method also includes storing and marking specific peptide chains and cutting sites on the protein sequence based on their scores. This process allows for the rapid identification and modification of potentially immunogenic peptide chains, facilitating the development of therapeutic proteins with reduced immunogenicity.

In addition to the method, the specification describes a computer system and a computer-readable storage medium designed to implement this method. The system comprises a processor and a memory storing a program executable by the processor to carry out the immunogenicity evaluation and modification process.

Further, this computer-assisted immunogenicity assessment method uses deep learning neural network algorithms to deeply learn the humanness. It constructs a predictive model for the humanness peptide chains, cuts the protein sequence into peptide chains of any potential length, and evaluates these chains in the predictive model to assess their immunogenicity. This method can completely generate all possible hydrolyzed peptide chain structures of a specific length from a protein sequence and then predict each chain's binding ability with MHC class alleles. The method is comprehensive, accurate, efficient, and accelerates the development of therapeutic proteins.

Furthermore, this specification also provides a solution where, after obtaining the humanness scores of peptide chains, it includes the following steps:
Saving peptide chains whose humanness scores with the preset T-cell epitope sequence are smaller than or equal to the first preset threshold; and/or
Marking the cutting sites on the protein sequence where the binding ability scores are smaller than or equal to the first preset threshold.

Another solution provided by this specification includes obtaining protein sequences by translating them into FASTA format files and importing these files into the predictive scoring model.

Yet another solution involves the data of the model training dataset, which includes data from the IEDB database.

Yet another aspect of the present invention is to provide a computer-assisted method for immunogenicity assessment, including the steps described in any of the above methods, obtaining peptide chains whose humanness scores are smaller than or equal to the second preset threshold, traversing all possible single-point virtual mutations of the chain to generate a set of modified peptide chains, and scoring the binding ability of each modified peptide chain with the preset T-cell epitope sequence.

After obtaining the humanness scores of the modified peptide chains, another solution involves replacing the original peptide chains with those whose scores are above the second preset threshold to create a modified protein sequence.

Another approach includes single-point virtual mutations involving amino acid scanning mutagenesis.

Additionally, the invention also provides a computer system comprising a memory, a processor, and a computer program stored in the memory and executable on the processor. When the processor executes the computer program, it implements the steps of the computer-assisted immunogenicity assessment method as described earlier in the specification.

Additionally, the invention provides a computer-readable storage medium storing a computer program, which, when executed by a processor, implements the steps of the computer-assisted immunogenicity assessment method as described earlier in the specification.

Compared to existing technology, at least one of the technical solutions adopted in the embodiments of this specification can achieve beneficial effects that include: using the computer-assisted immunogenicity assessment method, which does not rely on the experience of researchers, but uses computer simulation to generate all possible hydrolyzed peptide chain structures of a protein sequence, ensuring comprehensive screening. The training data input into the predictive scoring model covers a variety of different MHC allele and peptide chain binding data. Through the neural network model and rational training approach, it can accurately predict the binding ability of similar MHC and peptide molecules, expanding the prediction range, making the immunogenicity prediction of proteins more comprehensive. The applicability of this method is good. As new data on MHC allele and peptide chain binding accumulate, these new data can be used as training data for model training. The predictive scoring model can be quickly iterated, effectively covering the polymorphism of MHC, thereby accelerating the development of therapeutic proteins, significantly improving the efficiency and effectiveness of immunogenicity response assessment, and greatly reducing the time and screening costs of downstream experiments. Implementation of the Invention.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

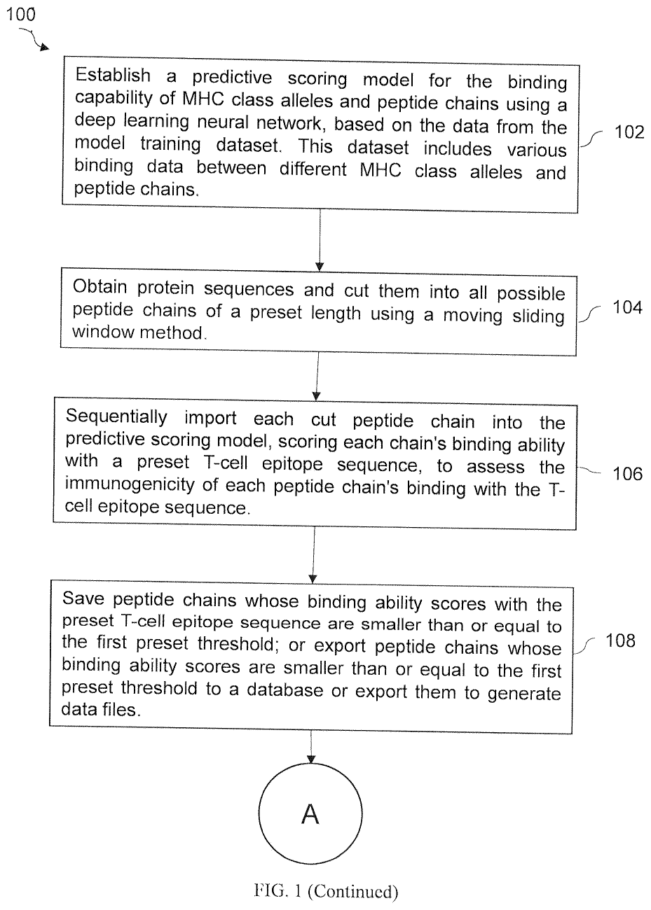

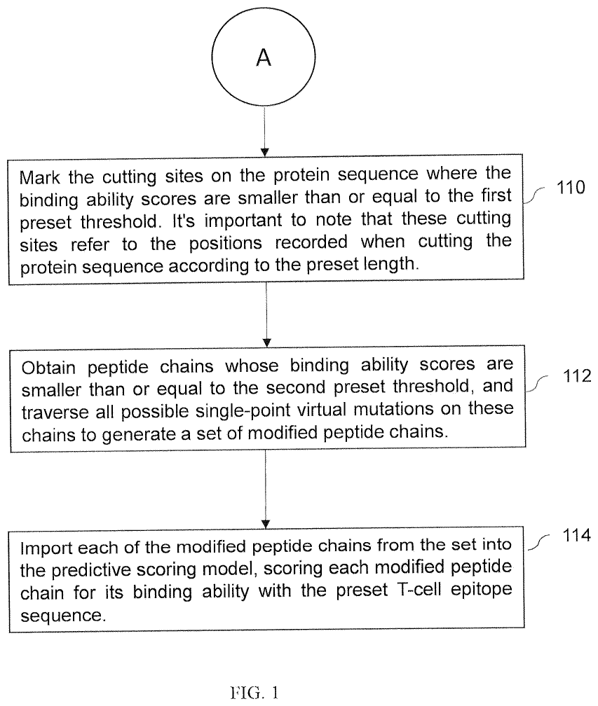

Referring now to the drawings, and more particularly to FIG. 1 a schematic flow diagram 100 for steps in a computer-assisted method for immunogenicity assessment, where similar reference characters denote corresponding features consistently throughout the figures, there are shown preferred embodiments, and these embodiments are described in the context of the following exemplary system and/or method.

FIG. 1 is a schematic flow diagram 100 for steps in a computer-assisted method for immunogenicity assessment, in accordance with an embodiment of the present disclosure for evaluating and modifying immunogenicity of protein sequences using a protein large language model, in accordance with an embodiment of the present disclosure.

According to FIG. 1, Step 102: Establish a predictive scoring model for the binding capability of MHC class alleles and peptide chains using a deep learning neural network, based on the data from the model training dataset. This dataset includes various binding data between different MHC class alleles and peptide chains. It's important to note that this binding data can come from open-source databases, commercial databases, or data reported in literature, or a combination thereof.

Step 104: Obtain protein sequences and cut them into all possible peptide chains of a preset length using a moving sliding window method. The preset length can be freely set according to the researchers' needs.

Step 106: Sequentially import each cut peptide chain into the predictive scoring model, scoring each chain's binding ability with a preset T-cell epitope sequence, to assess the immunogenicity of each peptide chain's binding with the T-cell epitope sequence. The trained predictive scoring model is used to score each peptide chain of a specific length for its binding ability with the T-cell epitope sequence, which can be one or multiple types.

In the above scheme, the neural network model and rational training approach can accurately predict the binding ability of similar MHC and peptide molecules, expanding the range of prediction. By establishing a predictive scoring model and accumulating new data as training data, the model can quickly iterate and effectively cover the polymorphism of MHC. The computer simulation generates all possible hydrolyzed peptide chain structures of a protein sequence, ensuring a comprehensive assessment of the protein sequence. This assessment method can accelerate the development of therapeutic proteins, significantly improve the efficiency and effectiveness of immunogenicity response assessment, and greatly reduce the time and cost of downstream experiments.

It should be noted that for the same protein sequence that needs prediction, steps 104 and 106 can be repeated multiple times to predict the immunogenicity scores of peptide chains of different lengths.

In some embodiments, after obtaining the humanness scores of the cut peptide chains with the preset T-cell epitope sequence, the computer-assisted immunogenicity assessment method includes:

Step 108: Save peptide chains whose binding ability scores with the preset T-cell epitope sequence are smaller than or equal to the first preset threshold; or export peptide chains whose binding ability scores are smaller than or equal to the first preset threshold to a database or export them to generate data files. The first preset threshold can be manually set or be a default system value.

Step 110: Mark the cutting sites on the protein sequence where the binding ability scores are smaller than or equal to the first preset threshold. It's important to note that these cutting sites refer to the positions recorded when cutting the protein sequence according to the preset length.

In the above scheme, by marking high-scoring sites on the original protein sequence, the immunogenicity response level of the protein sequence can be intuitively and clearly reflected.

In some embodiments, a computer system includes an interactive interface where protein sequences can be directly input, facilitating the acquisition of protein sequences in step 104.

In some embodiments, protein sequences are translated into FASTA format files, which are then imported into the predictive scoring model, thus facilitating the acquisition of protein sequences in step 104.

In some embodiments, the data of the model training dataset includes data from the IEDB database (Immune Epitope Database). It should be noted that the data for the model training dataset is not limited to the IEDB database but can also include data from other databases, literature, or other publicly disclosed sources.

Based on the same inventive concept, this invention also provides a computer-assisted method for immunogenicity modification. On the basis of using any of the above-mentioned computer-assisted immunogenicity assessment methods, peptide chains whose humanness scores are smaller than or equal to the second preset threshold are obtained. This computer-assisted immunogenicity modification method includes the following steps:

Step 112: Obtain peptide chains whose binding ability scores are smaller than or equal to the second preset threshold, and traverse all possible single-point virtual mutations on these chains to generate a set of modified peptide chains.

Step 114: Import each of the modified peptide chains from the set into the predictive scoring model, scoring each modified peptide chain for its binding ability with the preset T-cell epitope sequence.

Specifically, a computer system includes an interactive module, an interface for user input, where users mainly need to input the modified protein sequence, the modification threshold, and the task type. Protein sequences can be uploaded in FASTA file format or directly input as sequences; the modification threshold is a score between 0-1 representing the binding ability of peptide chains with MHCII, with higher scores indicating stronger binding ability, and the default modification threshold is 0.5.

Task types include analysis tasks and modification tasks, which can be selected in the interactive module. Analysis tasks only analyze the immunogenicity of user-input or uploaded sequences, i.e., performing steps 102 to 106, scoring each cut peptide chain, and marking on the original protein sequence where the immunogenicity of peptide chains exceeds the threshold after scoring; modification tasks, after completing the sequence immunogenicity analysis, modify the chains exceeding the threshold to achieve de-immunogenicity, i.e., performing steps 112 and 114. For example, for a given 140-length protein sequence, using a window length of 15 and a step length of 5, all possible peptide chains are cut using a dynamic sliding window method, and then each peptide chain is scored using the trained predictive scoring model to assess immunogenicity.

In the above scheme, the neural network trained predictive scoring model can accurately predict the binding ability of peptide chains and MHCII; by scoring each cut peptide chain, it attempts to screen as much as possible the peptide chains' binding ability with MHC; users can set the threshold, and when the binding ability of peptide chains with MHC exceeds the threshold range, the chains are modified via single-point virtual mutations to reduce their binding ability with MHC.

It should be noted that steps 112 and 114 here can also be combined with steps 110 and/or 108 in the above scheme to obtain more application schemes.

For example, first perform step 110, marking the cutting sites where the binding ability scores are smaller than or equal to the first preset threshold, then perform steps 112 and 114, scoring each modified peptide chain after single-point mutation using the predictive scoring model established in step 102. Additionally, in some specific embodiments, on the original protein sequence (i.e., before cutting), a method similar to step 110 can be used to mark the original cutting sites where the scores of the modified peptide chains exceed the second preset threshold. It should be noted that the second preset threshold in step 112 here can be the same as or different from the first preset threshold in step 110.

For another example, first perform step 108, saving the peptide chains whose humanness scores before modification are smaller than or equal to the first threshold, then perform steps 112 and 114 on these chains exceeding the first threshold to assess whether the binding ability of these chains is reduced after single-point mutation modification. It should be noted that the second preset threshold in step 112 here can be the same as or different from the first preset threshold in step 108.

In some embodiments, after performing step 114, replace the original peptide chains with the modified peptide chains whose scores are above the second preset threshold to create a modified protein sequence. In this scheme, for peptide chains with high immunogenicity, all possible single-point virtual mutations are traversed to find potentially immunogenicity-reducing peptide chains, choosing better scoring (lower scoring) chains to replace the original chains for modification. In some embodiments, after performing step 114, save the structure of the modified peptide chains whose humanness scores are above the second preset threshold.

In other embodiments, after performing step 114, the structure of the modified peptide chains whose humanness scores with the preset T-cell epitope sequence are smaller than or equal to the second preset threshold can also be saved, where the second preset threshold is equal to the first preset threshold.

In some embodiments, the single-point mutation method includes amino acid scanning mutagenesis, and it should be noted that the amino acid scanning mutagenesis includes using any amino acid for mutagenesis, not limited to alanine scanning mutagenesis.

In the above scheme, by setting a reasonable single-point mutation plan and then bringing the mutated peptide chains back into the predictive scoring model for binding ability assessment, it can prevent the mutation from causing other positions on the peptide chain to enhance binding ability with MHC, leading to changes in the overall immunogenicity of the entire protein sequence.

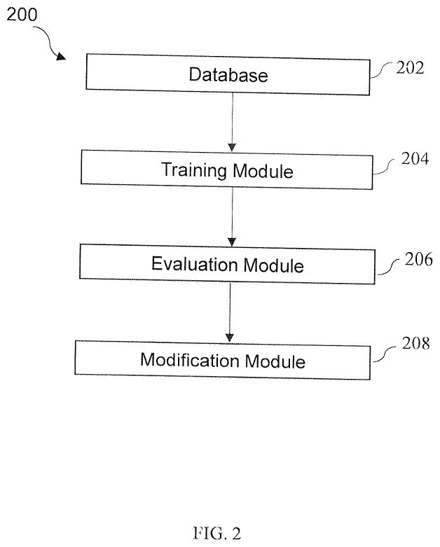

FIG. 2 is a detailed view of a system architecture 200, in accordance with the present disclosure.

According to FIG. 2, the system architecture 200, includes a database 202, training module 204, an evaluation module 206 and a modification module 208. The database 202 is configured to have a comprehensive collection of protein sequences and corresponding immunogenicity data. Further, the training module 204 is configured to utilize the database to train the PLLM on recognizing patterns associated with various levels of immunogenicity. The evaluation module 206 is further configured to have inputs of a protein sequence to the PLLM to predict its immunogenic potential. Furthermore, the modification module 208 is configured to suggest modifications to the protein sequence to achieve a desired immunogenic profile, based on the PLLM's predictions.

Based on the same inventive concept, the embodiments in this specification also provide a computer system, including a memory, a processor, and a computer program stored in the memory and executable on the processor. When the processor executes the computer program, it implements the steps of the computer-assisted immunogenicity assessment method as described in any of the aforementioned embodiments.

This text describes various embodiments of a computer system and a computer-readable storage medium that stores a computer program. The computer program, when executed by a processor, implements steps of a computer-assisted immunogenicity assessment method. The technical effects brought by the computer-readable storage medium are similar to those described in the previous embodiments of the computer-assisted immunogenicity assessment method and are not reiterated here.

### DETAILED DESCRIPTION OF THE EMBODIMENT

The implementation of the computer-assisted method for evaluating and modifying immunogenicity involves integrating advanced artificial intelligence techniques, specifically tailored for protein sequences.

Deep Learning Neural Network with Protein LLM: A key component is the unsupervised deep learning neural network that includes a protein Large Language Model (LLM).

This network integrates a variety of supervised learning architectures such as CNN, RNN, GNN, VAE and Transformer models, specifically designed for analyzing protein sequences between human and non-human species to generate a set of humanness scores.

Extensive Pre-Training: The protein LLM is pre-trained on a comprehensive dataset of over 3.5 billion species-specific protein sequences. This pre-training phase equips the model with an extensive understanding of protein sequences and functions.

Protein Sequence Processing: Protein sequences are processed using a dynamic window method to generate all possible peptide chains of a preset length. For antibodies, the peptide chains to be generated will preserve the CDR1, CDR2 and CDR3 regions of each chain. This exhaustive approach ensures that every potential immunogenic sequence within the protein is evaluated.

Evaluation and Modification of Peptide Chains: Each peptide chain is scored using the protein LLM for humanness score. Peptide chains with high immunogenic potential undergo single-point virtual mutations to generate modified chains, which are then re-evaluated using the model.

Comprehensive Evaluation Matrices: The model's performance is evaluated using a range of matrices such as ROC, AUC, Atom-IoU, Top(n), Top(n+2), Precision, and Recall, ensuring a thorough assessment of its predictive capabilities.

Computer System and Storage Medium: The method is implemented on a computer system equipped with a processor and memory. A computer-readable storage medium contains the program for executing the method, leveraging the advanced capabilities of the protein LLM in unsupervised and supervised learning tasks.

### EXAMPLES OF THE INVENTION

To further illustrate the invention, several examples are provided. These examples demonstrate the practical application and effectiveness of the method in evaluating and modifying the immunogenicity of protein sequences.

### Example 1: Evaluating Immunogenicity of Antibody Sequence

In this example, a specific protein sequence is processed using the computer-assisted method. The sequence is cut into peptide chains of a preset length between 6-12 amino acids without breaking the CDR1, CDR2 and CDR3 sequences. These chains are then scored for their humanness score using the predictive scoring model. Peptide chains with scores above a certain threshold of humanness score are identified as potentially immunogenic. This example demonstrates the method's ability to comprehensively screen a protein sequence for immunogenic peptides.

### Example 2: Modifying Immunogenic Peptide Chains

Building on Example 1, this example involves modifying the identified immunogenic peptide chains. Single-point virtual mutations are performed on these chains to generate a set of modified peptide chains. These modified chains are then re-scored using the predictive scoring model. Chains with increased scores, indicating lower immunogenicity, are selected. This example illustrates the method's capability to modify and reduce the immunogenicity of peptide chains.

### Example 3: Application in Therapeutic Protein Development

This example highlights the method's application in the development of a therapeutic protein. The protein sequence is screened for immunogenic peptides, and potentially immunogenic chains are modified to reduce their immunogenicity. The resulting protein is then subjected to further development and testing. This example showcases how the method can accelerate the development of therapeutic proteins with reduced risk of adverse immune reactions.

Conclusion and Future Prospects—This invention presents a novel and efficient approach to evaluating and modifying the immunogenicity of protein sequences. The use of a deep learning neural network to establish a predictive scoring model marks a significant advancement over traditional methods. This model's ability to rapidly and accurately predict the humanness scores is pivotal in identifying and modifying immunogenic peptide chains.

The comprehensive nature of this method, combined with its high accuracy and adaptability, makes it a valuable tool in the development of therapeutic proteins. It not only reduces the time and cost associated with immunogenicity testing but also enhances the safety and efficacy of the final therapeutic products.

Looking forward, the continued refinement and expansion of the training dataset will further improve the model's accuracy and adaptability. Additionally, the integration of this method into the broader workflow of therapeutic protein development can streamline the process, making it more efficient and effective.

This invention has the potential to significantly impact the field of biotechnology and pharmaceuticals, particularly in the development of safe and effective therapeutic proteins. Its application could extend beyond therapeutic proteins to other areas where protein immunogenicity is a concern, offering broad and far-reaching benefits.

Numerous advantages of the present disclosure may be apparent from the discussion above. In accordance with the present disclosure, several significant advantages are as follows:

Comprehensive Immunogenicity Screening: By generating all possible peptide chain sequences from a protein sequence, the method ensures comprehensive screening of potential immunogenic peptides. This is a significant improvement over traditional methods, which may miss certain immunogenic sequences.

High Accuracy and Adaptability: The deep learning model, trained with diverse data sources, can accurately predict the humanness scores of peptide chains to various species from human to non-human protein sequence information. This model is highly adaptable and can quickly incorporate new data, thus continuously improving its predictive accuracy.

Efficiency in Therapeutic Protein Development: The method significantly accelerates the development process of therapeutic proteins by rapidly identifying and modifying potentially immunogenic peptide chains. This reduces the time and resources needed for experimental immunogenicity testing.

Reduced Risk of Adverse Immune Reactions: By modifying peptide chains to reduce their immunogenicity, the method lowers the risk of adverse immune reactions in patients, increasing the safety and efficacy of therapeutic proteins.

Application in Personalized Medicine: The adaptability of the model to various MHC class alleles makes it suitable for personalized medicine applications, where individual differences in immune responses are considered.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

What is claimed is:

1. A computer-assisted method for evaluating immunogenicity of protein sequences using a protein large language model, characterized by:
establishing a predictive scoring model for the humanness score of protein sequences using a deep learning neural network, including a protein large language model, based on data from a model training dataset comprising various human and non-human species protein sequence information.
obtaining protein sequences and processing them into peptide chains of a preset length using a dynamic window method.
importing these peptide chains into the protein large language model for scoring each chain's humanness score, thereby evaluating their immunogenicity.

2. The method according to claim 1, further characterized by:
storing peptide chains whose humanness score, as determined by the protein large language model, are smaller than or equal to a first preset threshold; and/or
marking cutting sites on the protein sequence where the humanness scores are smaller than or equal to a first preset threshold.

3. The method according to claim 1, wherein obtaining protein sequences includes translating protein sequences into FASTA format files and importing these files into the protein large language model through tokenization.

4. The method according to claim 1, where the data of the model training dataset is processed through the protein large language model.

5. The method according to claim 1, wherein the protein large language model is part of a unsupervised deep learning model, which is integrated with a combination of supervised learning models including CNN, RNN, GNN, VAE and Transformer models, all tailored for analyzing protein sequences.

6. A computer-assisted method for modifying immunogenicity of protein sequences using a protein large language model, characterized by:
implementing the method for evaluating immunogenicity as described in claims 1 to 5;
identifying, via the protein large language model that includes CNN, RNN, GNN, VAE and Transformer models, peptide chains whose humanness scores are smaller than or equal to a second preset threshold;
performing all possible single-point virtual mutations on these identified peptide chains to generate a set of modified peptide chains;
scoring the humanness score of each modified peptide chain using the protein large language model that includes CNN, RNN, GNN, VAE and Transformer models; and
selecting modified peptide chains whose humanness scores are more than the second preset threshold.

7. A computer system for evaluating and/or modifying immunogenicity of protein sequences, characterized by including a processor and a memory connected to the processor, wherein the memory stores a program executable by the processor to implement the method for evaluating and/or modifying immunogenicity using a protein large language model, as described in any one of claims 1 to 6, wherein the protein large language model followed by a combination of CNN, RNN, GNN, VAE and Transformer models for comprehensive protein sequence analysis.

8. A computer-readable storage medium, characterized by storing a computer program, which, when executed by a processor, implements the method for evaluating and/or modifying immunogenicity of protein sequences using a protein large language model, according to any one of claims 1 to 6, wherein the protein large language model includes CNN, RNN, GNN, VAE and Transformer models as part of its deep learning architecture.
